(19) United States
(12) Patent Application Publication
(10) Pub. No.: US 2011/0262924 A1
(43) Pub. Date: Oct. 27, 2011

(54) MOLECULAR ASSAY FOR DIAGNOSIS OF HIV TROPISM

(75) Inventors: Mark A. Hayden, Ingleside, IL (US); Thomas G. Laffler, Libertyville, IL (US)

(73) Assignee: ABBOTT LABORATORIES, Abbott Park, IL (US)

(21) Appl. No.: 13/140,271

(22) PCT Filed: Dec. 18, 2009
(86) PCT No.: PCT/US09/68665

§ 371 Date: Jun. 16, 2011

Related U.S. Application Data
(60) Provisional application No. 61/139,418, filed on Dec. 19, 2008.

(51) Int. Cl. C12Q 1/68

(52) U.S. Cl. 435/6.12

(57) ABSTRACT

The invention is directed to compositions, methods and kits for HIV subtypes in a test sample, wherein target sequence are amplified. The amplified target sequences are then analyzed by any number of mass spectrometric techniques, which data are queried against a database of base composition signatures of HIV subtypes.

### FIELD OF THE INVENTION

The present invention is generally directed to detecting Human Immunodeficiency Virus (HIV) tropisms.

### BACKGROUND OF THE INVENTION

Human immunodeficiency virus (“HIV”) is a human retrovirus that is an etiologic agent of acquired immune deficiency syndrome (“AIDS”), an infectious disease characterized by a profound loss of immune system function. Two types of HIV are known, HIV-1 and HIV-2.

The globally circulating strains of HIV-1 exhibit extreme genetic diversity (Robertson et al., 1995). To evaluate the extent of global HIV-1 variation, sequences of virus strains originating from numerous countries have been compared. These studies have shown that HIV-1 can be classified into two major groups, namely, Group M (for “Major”) and Group 0 (for “Outlier”). HIV-1 Group M and Group 0 are distinct clusters on phylogenetic trees. Group M comprises the great majority of HIV-1 isolates and can be further subdivided into at least nine sequence subtypes or clades, designated A to I, with additional variants being added continually to the classification scheme (Gao et al., 1996). Although Group 0 strains are highly divergent, the clinical course of Group 0 infection is identical to that of HIV-1 Group M infection.

The main cells targeted by HIV-1 are T-cells, macrophages and dendritic cells (Clapham and McKnight, 2001). HIV-1 interacts with several receptors on the surfaces of these cells to trigger the fusion of viral and cellular membranes in order to confer virus entry into these cells (Clapham and McKnight, 2001). HIV-1 tropism refers to the types of cells that HIV-1 infects and the means by which infection is accomplished.

HIV-1 interacts with the CD4 glycoprotein and a seven transmembrane (7™) co-receptor to trigger entry into cells (Clapham and McKnight, 2001) HIV-1 contains envelope glycoprotein “spikes” on its surface that comprise an outer surface protein, gp120, that is non-covalently linked to a transmembrane protein, gp41. Each “spike” on an HIV-1 particle consists of a trimer of three gp120 proteins and three gp41 proteins (Clapham and McKnight, 2001). The binding of CD4 to gp120 triggers a structural change that exposes a binding site for a co-receptor (Clapham and McKnight, 2001). Further structural rearrangements are initiated when the co-receptor is bound. These changes are believed to be sufficient to trigger the fusion of viral and cellular membranes thus allowing entry of the virion core into the cytoplasm of a cell. (Clapham and McKnight, 2001).

Over fourteen different 7™ receptors have been identified as potential co-receptors for HIV-1. These receptors are either members of, or closely related to, the chemokine receptor family. Two major co-receptors of the chemokine receptor family are CCR5 and CXCR4. All HIV-1 isolates use one or both of these co-receptors. Specifically, the CCR5 receptor is the major co-receptor for macrophage-tropic (R5) strains, which play a crucial role in the sexual transmission of HIV-1. T cell line-tropic (X4) viruses use CXCR4 to enter target cells. CXCR4-using viruses tend to emerge in the later stages of infection in around 60% of progressing patients and their emergence coincides with accelerated disease progression (Westby et al., 2006). However, whether CXCR4 emergence is a cause or a consequence of severe immune system impairment is unknown, as little is known about the mechanism by which CXCR4 viruses are selected during the course of infection (Westby et al., 2006). Some HIV-1 isolates are dualtropic (R5×4) since they can use both co-receptors, although not always with the same efficiency.

The CCR5 and CXCR4 co-receptors are attractive targets for drug development since they are members of the G protein-coupled receptor superfamily, a group of proteins targeted by several commonly used and well-tolerated drugs, such as desloradine, ranitidine and tegaserod (Westby et al., 2006). CCR5 is of particular interest since a natural polymorphism exists in humans (CCR5-Δ32) that leads to reduced or absent cell surface expression of CCR5 in heterozygotic or homozygotic genotypes, respectively (Westby et al., 2006). Individuals homozygotic for CCR5-Δ32 appear to benefit from a natural resistance to HIV-1 infection, while heterozygotic CCR5-Δ32 is associated with reduced disease progression (Westby et al., 2006). Antagonists that block the binding of HIV-1 to the CCR5 are being developed as the first anti-HIV agents acting on a host target cell. However, these antagonists are only effective in patients lacking the CXCR4 co-receptor. Currently, there is a belief that selective inhibition of CCR5—using strains by treatment with CCR5 antagonists may lead to an increased rate of emergence of CXCR4 variants (Westby et al., 2006).

A number of assays for determining the tropism of an HIV-1 population are known in the art. These assays involve either determining the binding to receptors displayed on cell surfaces (phenotyping) or inferring tropism from genetic information. For example, U.S. Pat. No. 7,294,458 describes a phenotyping assay that involves transforming cells with an HIV envelope gene cloned from an infected patient, selectively fusing the cells with an indicator cell line that expresses an HIV envelope-compatible co-receptor and then assaying for fusion. Cell surface envelope protein variants selectively interact with either CCR5 or CXCR4 co-receptors. Fusion occurs only when an envelope protein interacts with a compatible co-receptor present on the surface of indicator cells. Cells expressing a particular envelope gene will fuse either CCR5 or CXCR4 indicator cells depending on the patient's envelope gene specificity. Fusion with either CCR5 or CXCR4 indicator cells indicates the type of co-receptor usage. U.S. Pat. No. 7,235,356 describes another phenotyping assay, wherein the disclosed method comprises (a) obtaining nucleic acid encoding a viral envelope protein from a patient infected by the virus; (b) co-transfecting into a first cell (i) the nucleic acid of step (a), and (ii) a viral expression vector that lacks a nucleic acid encoding an envelope protein and comprises an indicator nucleic acid which produces a detectable signal, such that the first cell produces viral particles comprising the envelope protein encoded by the nucleic acid obtained from the patient; (c) contacting the viral particles produced in step (b) with a second cell in the presence of the compound, wherein the second cell expresses a cell surface receptor to which the virus binds; (d) measuring the amount of signal produced by the second cell in order to determine the infectivity of the viral particles; and (e) comparing the amount of signal measured in step (d) with the amount of signal produced in the absence of the compound, wherein a reduced amount of signal measured in the presence of the compound indicates that the compound inhibits entry of the virus into the second cell. Van Baelen et al also describe another phenotyping assay (Van Baelen et al., 2007). US Patent Application Publication No. US2006/0194227 describes a heteroplex assay wherein (a) HIV viral RNA is obtained from the patient; (b) a portion of the viral genome containing genetic determinates of co-receptor usage, e.g. a genomic portion comprising the V3 domain of the gp120 envelope glycoprotein, is amplified; (c) heteroduplexes and/or homoduplexes are formed with labeled nucleic acid-based probes prepared from a corresponding genomic region of a known HIV strain; and (d) the heteroduplexes and homoduplexes are separated (such as on a gel), resulting in characteristic mobility patterns such that the coreceptor usage can be determined. As is apparent from the description of these assays, they are time consuming and expensive.

The introduction of CCR5 antagonists, such as Marovic (4,4-difluoro-N-$(1S)-3-[3-(3-isopropyl-5-methyl-4H-1,2,4-triazol-4-yl)-8-azabicyclo[3.2.1]oct-8-yl]-1-phenylpropyl$cyclohexanecarboxamide, increases the options available for constructing antiretroviral regimens. However, this option is coupled with the caveat that patients should be tested for HIV co-receptor tropism prior to initiating CCR5 antagonist-based therapy. Failure to screen for CXCR4 usage increases the risk of using an ineffective drug, thus reducing the likelihood of viral suppression and increasing risk for developing antiretroviral resistance (Low et al., 2008).

There is a need in the art for assays for determining the tropism of an HIV population that can be performed rapidly, allowing for screening patients as suitable candidates for CXCR4 or CCR5 HIV subtypes therapy.

Nucleic Acid-Based Molecular Diagnostics

Molecular diagnostics have been championed for identifying pathogens. Polymerase chain reaction (PCR)-based diagnostics, wherein target polynucleotide sequences are amplified in vitro and then detected, have been successfully developed for a wide variety of pathogens.

The principal shortcomings of applying PCR assays to the clinical setting include the inability to eliminate background DNA contamination, interference with the PCR amplification by competing substrates, and limited capacity to discern speciation, antibiotic resistance and pathogen subtype. Despite significant progress, contamination remains problematic, and methods directed towards eliminating exogenous sources of DNA often also result in significant diminution in assay sensitivity. Although simple DNA sequencing can be performed to identify and characterize PCR products, sequencing and the subsequent analysis can be laborious and time-consuming.

Mass spectrometric techniques, such as high resolution electrospray ionization-Fourier transform-ion cyclotron resonance mass spectrometry (ESI-FT-ICR MS), can be used for quick PCR product detection and characterization. Accurate measurement of the exact mass combined with knowledge of the number of at least one nucleotide allows for calculating the total base composition for PCR duplex products of approximately 100 base pairs (Muddiman and Smith, 1998). For example, Aaserud et al demonstrated that accurate mass measurements obtained by high-performance mass spectrometry can be used to derive base compositions from double-stranded synthetic DNA constructs using the mathematical constraints imposed by the complementary nature of the two strands (Aaserud et al., 1996). Muddiman et al. developed an algorithm that allowed for deriving unambiguous base compositions from the exact mass measurements of the complementary single-stranded oligonucleotides (Muddiman et al., 1997). Wunschel et al showed that PCR products amplified from templates differing by a single nucleotide can be resolved and identified using ESI-FTICR at the 89-bp level in PCR product amplified from a 16/23S rDNA interspace region from Bacillus cereus (Wunschel et al., 1998). Electrospray ionization-Fourier transform-ion cyclotron resistance (ESI-FT-ICR) MS can be used to determine the mass of double-stranded, 500 base-pair PCR products via the average molecular mass (Hurst et al., 1996). The use of matrix-assisted laser desorption ionization-time of flight (MALDI-TOF) mass spectrometry for characterizing PCR products has also been exploited (Muddiman et al., 1999).

Examples of mass spectrometric analysis of polynucleotides include:

U.S. Pat. No. 5,965,363 discloses methods for screening nucleic acids for polymorphisms by analyzing amplified target nucleic acids using mass spectrometric techniques and procedures for improving mass resolution and mass accuracy of these methods.

WO 99/14375 describes methods, PCR primers and kits for use in analyzing preselected DNA tandem nucleotide repeat alleles by mass spectrometry.

WO 98/12355 discloses methods of determining the mass of a target nucleic acid by mass spectrometric analysis, by cleaving the target nucleic acid to reduce its length, making the target single-stranded and using MS to determine the mass of the single-stranded shortened target. Also disclosed are methods of preparing a double-stranded target nucleic acid for MS analysis comprising amplification of the target nucleic acid, binding one of the strands to a solid support, releasing the second strand and then releasing the first strand which is then analyzed by MS. Kits for target nucleic acid preparation are also provided.

PCT WO97/33000 discloses methods for detecting mutations in a target nucleic acid by non-randomly fragmenting the target into a set of single-stranded nonrandom length fragments and determining their masses by MS.

U.S. Pat. No. 5,605,798 describes a fast and highly accurate mass spectrometer-based process for detecting the presence of a particular nucleic acid in a biological sample for diagnostic purposes.

WO 98/21066 describes processes for determining the sequence of a particular target nucleic acid by mass spectrometry. Processes for detecting a target nucleic acid present in a biological sample by PCR amplification and mass spectrometry detection are disclosed, as are methods for detecting a target nucleic acid in a sample by amplifying the target with primers that contain restriction sites and tags, extending and cleaving the amplified nucleic acid, and detecting the presence of extended product, wherein the presence of a DNA fragment of a mass different from wild-type is indicative of a mutation. Methods of sequencing a nucleic acid via mass spectrometry methods are also described.

WO 97/37041, WO 99/31278 and U.S. Pat. No. 5,547,835 describe methods of sequencing nucleic acids using mass spectrometry. U.S. Pat. Nos. 5,622,824, 5,872,003 and 5,691,141 describe methods, systems and kits for exonuclease-mediated mass spectrometric sequencing.

U.S. Pat. Nos. 7,217,510, 7,108,974, 7,255,992, 7,226,739 and US 2004/0219517 describe methods and compositions for identifying one or more bioagents that use at least one pair of oligonucleotide primers, wherein one pair hybridizes to two distinct conserved regions of a nucleic acid encoding a bioagent's ribosomal RNA, wherein the two distinct conserved regions flank a variable nucleic acid region that when amplified creates a base composition “signature” that is characteristic of the bioagents. The base composition signature, the exact base composition determined from the molecular mass of the amplified product, is determined by first determining the molecular mass of the amplification product by mass spectrometry, after which the base composition is determined from the molecular mass. The bioagent is determined by matching the base composition signature to those stored in a database.

### SUMMARY OF THE INVENTION

In a first aspect, the invention is directed to methods of identifying an HIV subtype in a test sample, comprising:

providing a test sample;

forming a reaction mixture comprising a primer pair set selected from the group consisting of set A, B, C, D, E, and F, wherein:
set A comprises a forward primer comprising a nucleic acid sequence of SEQ ID NO:1, and a reverse primer comprising a nucleic acid sequence of SEQ ID NO:2
set B comprises a forward primer comprising a nucleic acid sequence of SEQ ID NO:3, and a reverse primer comprising a nucleic acid sequence of SEQ ID NO:4; and
set C comprises a forward primer comprising a nucleic acid sequence of SEQ ID NO:5, and a reverse primer comprising a nucleic acid sequence of SEQ ID NO:6;
set D comprises a forward primer comprising a nucleic acid sequence of SEQ ID NO:7, and a reverse primer comprising a nucleic acid sequence of SEQ ID NO:8;
set E comprises a forward primer comprising a nucleic acid sequence of SEQ ID NO:9, and a reverse primer comprising a nucleic acid sequence of SEQ ID NO:10; and
set F comprises a forward primer comprising a nucleic acid sequence of SEQ ID NO:11, and a reverse primer comprising a nucleic acid sequence of SEQ ID NO:12;

subjecting the mixture to amplification conditions to generate an amplification product;

determining the molecular mass of the amplification product; and

comparing the molecular mass of the amplification product to calculated or measured molecular masses of target sequences in a database to identify the HIV subtype.

In a second aspect, the invention is directed to methods of identifying an HIV subtype in a test sample, comprising:

providing a test sample;

forming a reaction mixture comprising a primer pair set selected from the group consisting of set A, B, C, D, E, and F, wherein:
set A comprises a forward primer comprising a nucleic acid sequence of SEQ ID NO:1, and a reverse primer comprising a nucleic acid sequence of SEQ ID NO:2
set B comprises a forward primer comprising a nucleic acid sequence of SEQ ID NO:3, and a reverse primer comprising a nucleic acid sequence of SEQ ID NO:4; and
set C comprises a forward primer comprising a nucleic acid sequence of SEQ ID NO:5, and a reverse primer comprising a nucleic acid sequence of SEQ ID NO:6;
set D comprises a forward primer comprising a nucleic acid sequence of SEQ ID NO:7, and a reverse primer comprising a nucleic acid sequence of SEQ ID NO:8;
set E comprises a forward primer comprising a nucleic acid sequence of SEQ ID NO:9, and a reverse primer comprising a nucleic acid sequence of SEQ ID NO:10; and
set F comprises a forward primer comprising a nucleic acid sequence of SEQ ID NO:11, and a reverse primer comprising a nucleic acid sequence of SEQ ID NO:12;

subjecting the mixture to amplification conditions to generate an amplification product;

determining the base composition of the amplification product; and

comparing the base composition of the amplification product to calculated or measured base compositions of target sequences in a database to identify an HIV subtype.

In either of the methods of the first and second aspects, identifying the target sequence does not comprise sequencing of the amplification product, and the mass spectrometry can be Fourier transform ion cyclotron resonance mass spectrometry (FT-ICR-MS) or time of flight mass spectrometry (TOF-MS), such as electrospray ionization time of flight mass spectrometry (ESI-TOF). Additionally, in either of the first and second aspects, the primer set can comprise at least one nucleotide analog, wherein the nucleotide analog is, for example, inosine, uridine, 2,6-diaminopurine, propyne C, and propyne T, and the reaction mixture comprises at least two primer sets. The amplification products of both aspects can further comprise incorporating a molecular mass-modifying tag, such as an isotope of carbon, for example, ¹³C.

In a third aspect, the invention is directed to methods of identifying an HIV subtype in a test sample, comprising:

providing a test sample;

forming a reaction mixture comprising:

a primer pair set selected from the group consisting of set A, B, C, D, E, and F, wherein:
set A comprises a forward primer comprising a nucleic acid sequence having at least 80% sequence identity with a nucleic acid sequence of SEQ ID NO:1, and a reverse primer comprising a nucleic acid sequence having at least 80% sequence identity with a nucleic acid sequence of SEQ ID NO:2;
set B comprises a forward primer comprising a nucleic acid sequence having at least 80% sequence identity with a nucleic acid sequence of SEQ ID NO:3, and a reverse primer comprising a nucleic acid sequence having at least 80% sequence identity with a nucleic acid sequence of SEQ ID NO:4; and
set C comprises a forward primer comprising a nucleic acid sequence having at least 80% sequence identity with a nucleic acid sequence of SEQ ID NO:5, and a reverse primer comprising a nucleic acid sequence having at least 80% sequence identity with a nucleic acid sequence of SEQ ID NO:6;
set D comprises a forward primer comprising a nucleic acid sequence having at least 80% sequence identity with a nucleic acid sequence of SEQ ID NO:7, and a reverse primer comprising a nucleic acid sequence having at least 80% sequence identity with a nucleic acid sequence of SEQ ID NO:8;
set E comprises a forward primer comprising a nucleic acid sequence having at least 80% sequence identity with a nucleic acid sequence of SEQ ID NO:9, and a reverse primer comprising a nucleic acid sequence having at least 80% sequence identity with a nucleic acid sequence of SEQ ID NO:10; and
set F comprises a forward primer comprising a nucleic acid sequence having at least 80% sequence identity with a nucleic acid sequence of SEQ ID NO:11, and a reverse primer comprising a nucleic acid sequence having at least 80% sequence identity with a nucleic acid sequence of SEQ ID NO:12;

subjecting the mixture to amplification conditions to generate an amplification product;

determining the molecular mass of the amplification product; and

comparing the molecular mass of the amplification product to calculated or measured molecular masses of target sequences in a database to identify identifying an HIV subtype.

In a fourth aspect, the invention is directed to methods of identifying an HIV subtype in a test sample, comprising:

providing a test sample;

forming a reaction mixture comprising:

a primer pair set selected from the group consisting of set A, B, C, D, E, and F, wherein:
set A comprises a forward primer comprising a nucleic acid sequence having at least 80% sequence identity with a nucleic acid sequence of SEQ ID NO:1, and a reverse primer comprising a nucleic acid sequence having at least 80% sequence identity with a nucleic acid sequence of SEQ ID NO:2;
set B comprises a forward primer comprising a nucleic acid sequence having at least 80% sequence identity with a nucleic acid sequence of SEQ ID NO:3, and a reverse primer comprising a nucleic acid sequence having at least 80% sequence identity with a nucleic acid sequence of SEQ ID NO:4; and
set C comprises a forward primer comprising a nucleic acid sequence having at least 80% sequence identity with a nucleic acid sequence of SEQ ID NO:5, and a reverse primer comprising a nucleic acid sequence having at least 80% sequence identity with a nucleic acid sequence of SEQ ID NO:6;
set D comprises a forward primer comprising a nucleic acid sequence having at least 80% sequence identity with a nucleic acid sequence of SEQ ID NO:7, and a reverse primer comprising a nucleic acid sequence having at least 80% sequence identity with a nucleic acid sequence of SEQ ID NO:8;
set E comprises a forward primer comprising a nucleic acid sequence having at least 80% sequence identity with a nucleic acid sequence of SEQ ID NO:9, and a reverse primer comprising a nucleic acid sequence having at least 80% sequence identity with a nucleic acid sequence of SEQ ID NO:10; and
set F comprises a forward primer comprising a nucleic acid sequence having at least 80% sequence identity with a nucleic acid sequence of SEQ ID NO:11, and a reverse primer comprising a nucleic acid sequence having at least 80% sequence identity with a nucleic acid sequence of SEQ ID NO:12;

subjecting the mixture to amplification conditions to generate an amplification product;

determining the base composition of the amplification product; and

comparing the base composition of the amplification product to calculated or measured base compositions of target sequences in a database to identify identifying an HIV subtype.

In either of the methods of the third and fourth aspects, identifying the target sequence does not comprise sequencing of the amplification product, and the mass spectrometry is Fourier transform ion cyclotron resonance mass spectrometry (FT-ICR-MS) or time of flight mass spectrometry (TOF-MS), such as electrospray ionization time of flight mass spectrometry. Additionally, in either of the third and fourth aspects, the primer set can comprise at least one nucleotide analog, wherein the nucleotide analog is, for example, inosine, uridine, 2,6-diaminopurine, propyne C, and propyne T, and the reaction mixture comprises at least two primer sets. The amplification products of both aspects can further comprise incorporating a molecular mass-modifying tag

In a fifth aspect, the invention is directed to kits, comprising a primer pair set selected from the group consisting of set A, B, C, D, E, and F, wherein:
set A comprises a forward primer comprising a nucleic acid sequence of SEQ ID NO:1, and a reverse primer comprising a nucleic acid sequence of SEQ ID NO:2
set B comprises a forward primer comprising a nucleic acid sequence of SEQ ID NO:3, and a reverse primer comprising a nucleic acid sequence of SEQ ID NO:4; and
set C comprises a forward primer comprising a nucleic acid sequence of SEQ ID NO:5, and a reverse primer comprising a nucleic acid sequence of SEQ ID NO:6;
set D comprises a forward primer comprising a nucleic acid sequence of SEQ ID NO:7, and a reverse primer comprising a nucleic acid sequence of SEQ ID NO:8;
set E comprises a forward primer comprising a nucleic acid sequence of SEQ ID NO:9, and a reverse primer comprising a nucleic acid sequence of SEQ ID NO:10; and
set F comprises a forward primer comprising a nucleic acid sequence of SEQ ID NO:11, and a reverse primer comprising a nucleic acid sequence of SEQ ID NO:12; and

amplification reagents.

In a sixth aspect, the invention is directed to kits, comprising a primer pair set selected from the group consisting of set A, B, C, D, E, and F, wherein:
set A comprises a forward primer comprising a nucleic acid sequence having at least 80% sequence identity with a nucleic acid sequence of SEQ ID NO:1, and a reverse primer comprising a nucleic acid sequence having at least 80% sequence identity with a nucleic acid sequence of SEQ ID NO:2;
set B comprises a forward primer comprising a nucleic acid sequence having at least 80% sequence identity with a nucleic acid sequence of SEQ ID NO:3, and a reverse primer comprising a nucleic acid sequence having at least 80% sequence identity with a nucleic acid sequence of SEQ ID NO:4; and
set C comprises a forward primer comprising a nucleic acid sequence having at least 80% sequence identity with a nucleic acid sequence of SEQ ID NO:5, and a reverse primer comprising a nucleic acid sequence having at least 80% sequence identity with a nucleic acid sequence of SEQ ID NO:6;
set D comprises a forward primer comprising a nucleic acid sequence having at least 80% sequence identity with a nucleic acid sequence of SEQ ID NO:7, and a reverse primer comprising a nucleic acid sequence having at least 80% sequence identity with a nucleic acid sequence of SEQ ID NO:8;
set E comprises a forward primer comprising a nucleic acid sequence having at least 80% sequence identity with a nucleic acid sequence of SEQ ID NO:9, and a reverse primer comprising a nucleic acid sequence having at least 80% sequence identity with a nucleic acid sequence of SEQ ID NO:10;
set F comprises a forward primer comprising a nucleic acid sequence having at least 80% sequence identity with a nucleic acid sequence of SEQ ID NO:11, and a reverse primer comprising a nucleic acid sequence having at least 80% sequence identity with a nucleic acid sequence of SEQ ID NO:12; and

amplification reagents.

In another aspect, the invention is directed to methods of identifying at least two HIV subtypes in a test sample, comprising:

providing a test sample;

forming a reaction mixture comprising:

a primer pair set selected from the group consisting of set A, B, C, D, E, and F, wherein:
set A comprises a forward primer comprising a nucleic acid sequence having at least 80% sequence identity with a nucleic acid sequence of SEQ ID NO:1, and a reverse primer comprising a nucleic acid sequence having at least 80% sequence identity with a nucleic acid sequence of SEQ ID NO:2;
set B comprises a forward primer comprising a nucleic acid sequence having at least 80% sequence identity with a nucleic acid sequence of SEQ ID NO:3, and a reverse primer comprising a nucleic acid sequence having at least 80% sequence identity with a nucleic acid sequence of SEQ ID NO:4; and
set C comprises a forward primer comprising a nucleic acid sequence having at least 80% sequence identity with a nucleic acid sequence of SEQ ID NO:5, and a reverse primer comprising a nucleic acid sequence having at least 80% sequence identity with a nucleic acid sequence of SEQ ID NO:6;
set D comprises a forward primer comprising a nucleic acid sequence having at least 80% sequence identity with a nucleic acid sequence of SEQ ID NO:7, and a reverse primer comprising a nucleic acid sequence having at least 80% sequence identity with a nucleic acid sequence of SEQ ID NO:8;
set E comprises a forward primer comprising a nucleic acid sequence having at least 80% sequence identity with a nucleic acid sequence of SEQ ID NO:9, and a reverse primer comprising a nucleic acid sequence having at least 80% sequence identity with a nucleic acid sequence of SEQ ID NO:10; and
set F comprises a forward primer comprising a nucleic acid sequence having at least 80% sequence identity with a nucleic acid sequence of SEQ ID NO:11, and a reverse primer comprising a nucleic acid sequence having at least 80% sequence identity with a nucleic acid sequence of SEQ ID NO:12;

subjecting the mixture to amplification conditions to generate an amplification product;

determining the molecular mass of the amplification product; and

comparing the molecular mass of the amplification product to calculated or measured molecular masses of target sequences in a database to identify the at least two HIV subtypes, or comparing the base composition of the amplification product to calculated or measured base compositions of target sequences in a database to identify the at least two HIV subtypes. The two HIV subtypes can comprises CCR5-binding HIV and CXCR4-binding HIV; the CXCR4-binding HIV can comprises at least 1%, more than 1%, more than 5% and more than 10% of the total HIV in the test sample.

### BRIEF DESCRIPTION OF THE DRAWING

Not applicable

### DETAILED DESCRIPTION

Modern typical molecular assays to detect HIV variants typically use distinct molecular probes to identify the HIV subtype(s) present in test specimens. The present invention allows HIV subtypes to be determined based on amplicon base composition alone, thereby eliminating the need for species-specific molecular probes and labels. The invention thus simplifies detection in that fewer assay reagents—and effort—are required. Other advantages include the ability to detect multiple HIV subtypes simultaneously, reduced false positive and negative results due to the ability to screen multiple causative agents simultaneously, and speed. The invention thus allows for predicting HIV tropism, such that patients that are CCR5-exclusively tropic can be distinguished from CXCR4 tropic patients.

The invention discloses a method of predicting HIV tropism based on using mass spectrometry to determine the base composition of nucleic acid amplification products that contain the genetic region determining tropism. Primers are described that flank the tropism-determining domain within the HIV envelope gene and are capable of amplifying this region from a wide rage of HIV subtypes. Changes in the base composition of these amplification products are predictive of changes in HIV tropism.

The invention accomplishes its significant advantages in part by exploiting spectrometric technologies. For example, ElectroSpray Injection Time-of-Flight Mass Spectrometry (ESI-MS) can be used to determine the exact base composition of amplicons generated by target amplification technologies such as the polymerase chain reaction (PCR). The disclosed invention exploits primer pairs (sets A-F) directed to conserved regions that flank V3 loop variants of HIV gp120 envelope protein that are involved in tropism. The primers of the invention can be used to amplify DNA from any of the HIV variants. Base composition analysis by ESI-MS can then be used to identify which variant is (or are) present in a test sample.

The primer pair sets of the invention, sets A-F, are shown in Table 1. In one embodiment, the invention uses the novel primer pair of SEQ ID NOs:1 and 2 (Set A). In another embodiment, the invention uses the novel primer pair of SEQ ID NOs:3 and 4 (Set B). In another embodiment, the invention uses the novel primer pair of SEQ ID NOs:5 and 6 (Set C). In another embodiment, the invention uses the novel primer pair of SEQ ID NOs:7 and 8 (Set D) In another embodiment, the invention uses the novel primer pair of SEQ ID NOs:9 and 10 (Set D) In another embodiment, the invention uses the novel primer pair of SEQ ID NOs:11 and 12 (Set E). Table 2 gives examples of amplified sequences using primer pair sets A-C when the template is HIV-1 (SEQ ID NO:13; GenBank Accession No. AF033819; the gp160 gene, from which the gp120 polypeptide is processed, is located at nucleotides 5771-8341). Table 2 shows the amplified sequences when the primer pair sets A-C are used to amplify sequences from HIV-1; Table 3 shows only the target sequences amplified by primer pair sets D-F, as the primers are intended for base pair mis-matching during initial amplifications (see the Examples).

TABLE 1
Primer sequences

| | | SEQ | | Ampli- |
|---|---|---|---|---|
| | | ID | | con |
| Primer | Sequence | NO | Tm | length* |
| A (Forward) | aagacccaac aacaatacaa | 1 | 65° | |
| | ga | | | |
| A (Reverse) | gttacaatgt gcttgtctca | 2 | 65° | 106 |
| | tattt | | | |
| B (Forward) | aagacccaac aacaatacaa | 3 | 66° | |
| | gaa | | | |
| B (Reverse) | tgtgcttgtc tcatatttcc | 4 | 67° | 99 |
| | tattt | | | |
| C (Forward) | taattgtaca agacccaaca | 5 | 66° | |
| | aca | | | |
| C (Reverse) | atgtgcttgt ctcatatttc | 6 | 66° | 109 |
| | ctatt | | | |
| D (Forward) | gacccaacaa caatacaaga | 7 | 64° | |
| | a | | | |
| D (Reverse) | tgtgcttgtc ttatatctcc | 8 | 63° | 94 |
| | tatta | | | |
| E (Forward) | ttgtacaaga cccaacaaca | 9 | 64° | |
| E (Reverse) | aatgtgcttg tcttatatct | 10 | 63° | 104 |
| | cctat | | | |
| F (Forward) | attaattgta caagacccaa | 11 | 62° | |
| | ca | | | |
| F (Reverse) | atgtgcttgt cttatatctc | 12 | 63° | 108 |
| | ctatt | | | |

*Amplicon length includes primer sequences.

TABLE 2
Sequences amplified by primer sets A-C when HIV-1
is template (SEQ ID NO: 13)*

| Primer | | SEQ ID |
|---|---|---|
| Set | Sequence | NO: |
| A | aagacccaac aacaatacaa gaaaaagaat | 14 |
| | ccgtatccag agaggaccag ggagagcatt | |
| | tgttacaata ggaaaaatag gaaatatgag | |
| | acaagcacat tgtaac | |
| B | aagacccaac aacaatacaa gaaaaagaat | 15 |
| | ccgtatccag agaggaccag ggagagcatt | |
| | tgttacaata ggaaaaatag gaaatatgag | |
| | acaagcaca | |
| C | taattgtaca agacccaaca acaatacaag | 16 |
| | aaaaagaatc cgtatccaga gaggaccagg | |
| | gagagcattt gttacaatag gaaaaatagg | |
| | aaatatgaga caagcacat | |

*Amplicon sequence includes primer sequences.

TABLE 3
Target sequences amplified by primer sets D-F
when HIV-1 is template (SEQ ID NO: 13)**

| Primer | | SEQ ID |
|---|---|---|
| Set | Sequence | NO: |
| D | aaagaatccg tatccagaga ggaccaggga | 17 |
| | gagcatttgt tacaatagga a | |
| E | atacaagaaa aagaatccgt atccagagag | 18 |
| | gaccagggag agcatttgtt acaataggaa | |
| | aa | |
| F | acaatacaag aaaaagaatc cgtatccaga | 19 |
| | gaggaccagg gagagcattt gttacaatag | |
| | gaaa | |

**Reported sequence does not include primer sequences.

Since HIV is a retrovirus, having a single-stranded RNA genome, the target sequences are first reverse transcribed. The primers of the invention can be used in this step. Reverse-transcription protocols are well-known (Ausubel et al., 1987).

In another embodiment, the primer sets are subjected to amplification conditions, wherein the first cycle comprises incubating the reaction mix with at least one nucleic acid polymerase, such as a DNA polymerase, at 94° C. for 10 seconds, followed by 55-60° C. for 20 seconds, and then 72° C. for 20 seconds. The cycle can be repeated multiple times, such as for 35 cycles. A final cycle can be added, wherein the reaction mix is held at 4° C. In one embodiment, these conditions are applied when using a primer pair set of A, B or C. In another embodiment, low stringency amplification conditions, followed by high stringency amplification conditions are used, wherein the amplification mixture is subject to the following conditions: 94° C. for 10 seconds, followed by 40-50° C. for 20 seconds, and then 72° C. for 20 seconds for approximately 5 cycles, followed by 94° C. for 10 seconds, followed by 55-60° C. for 20 seconds, and then 72° C. for 20 seconds, for approximately 30 cycles. The latter amplification protocol, in one embodiment, is used with primer pair sets D, E, or F. These conditions can be adjusted as necessary, and easily, by one of skill in the art.

After amplification, the reaction mix is subjected to spectrometric analysis, such as ESI-MS. The sample is injected into a spectrometer, the molecular mass or corresponding “base composition signature” (BCS) of any amplification product is then determined and matched against a database of molecular masses or BCS's. A BCS is the exact base composition determined from the molecular mass of a bioagent identifying amplicon. BCS's provide a useful index of a specific gene in a specific organism. A BCS differs from nucleic acid sequence in that the signature does not order the bases, but instead represents the nucleic acid base composition of the nucleic acid (e.g., A, G, C, T). The present method thus provides rapid throughput and does not require nucleic acid sequencing of the amplified target sequence for detection and identification. Furthermore, time-consuming separation technologies, such as gel electrophoresis, coupled with detection of the separated sequences, whether from simple gel staining or hybridization with a probe comprising a detectable label, is avoided. In the methods of the invention, all HIV subtypes can be detected in a sample with a simple detection step and database interrogation.

In one embodiment, samples are obtained from a subject, which can be a mammal, such as a human. The sample is typically blood, but can be any other tissues that can harbor HIV.

### DEFINITIONS

“Specifically hybridize” refers to the ability of a nucleic acid to bind detectably and specifically to a second nucleic acid. Polynucleotides specifically hybridize with target nucleic acid strands under hybridization and wash conditions that minimize appreciable amounts of detectable binding by non-specific nucleic acids.

“Target sequence” or “target nucleic acid sequence” means a nucleic acid sequence of HIV or complements thereof, that is amplified, detected, or both using one or more of the polynucleotide primer sets of SEQ ID NOs:1 and 2, SEQ ID NOs:3 and 4, SEQ ID NOs:5 and 6, SEQ ID NOs:7 and 8, SEQ ID NOs:9 and 10, and SEQ ID NOs:11 and 12. Additionally, while the term target sequence sometimes refers to a double stranded nucleic acid sequence; a target sequence can also be single-stranded. In cases where the target is double-stranded, polynucleotide primer sequences of the present invention preferably amplify both strands of the target sequence. A target sequence can be selected that is more or less specific for a particular organism. For example, the target sequence can be specific to an entire genus, to more than one genus, to a species or subspecies, serogroup, auxotype, serotype, strain, isolate or other subset of organisms.

“Test sample” means a sample taken from an organism, including mosquitoes, or a biological fluid, wherein the sample may contain an HIV target sequence. A test sample can be taken from any source, for example, tissue, blood, saliva, sputa, mucus, sweat, urine, urethral swabs, cervical swabs, urogenital or anal swabs, conjunctival swabs, ocular lens fluid, cerebral spinal fluid, etc. A test sample can be used (i) directly as obtained from the source; or (ii) following a pre-treatment to modify the character of the sample. Thus, a test sample can be pre-treated prior to use by, for example, preparing plasma or serum from blood, disrupting cells or viral particles, preparing liquids from solid materials, diluting viscous fluids, filtering liquids, adding reagents, purifying nucleic acids, etc. Typically, test samples contain blood.

“Subjects” include a mammal, a bird, or a reptile. The subject can be a cow, horse, dog, cat, or a primate. The biological entity can also be a human. The biological entity may be living or dead.

A “polynucleotide” is a nucleic acid polymer of ribonucleic acid (RNA), deoxyribonucleic acid (DNA), modified RNA or DNA, or RNA or DNA mimetics (such as PNAs), and derivatives thereof, and homologues thereof. Thus, polynucleotides include polymers composed of naturally occurring nucleobases, sugars and covalent inter-nucleoside (backbone) linkages as well as polymers having non-naturally-occurring portions that function similarly. Such modified or substituted nucleic acid polymers are well known in the art and for the purposes of the present invention, are referred to as “analogues.” Oligonucleotides are generally short polynucleotides from about 10 to up to about 160 or 200 nucleotides.

“Variant polynucleotide” or “variant nucleic acid sequence” means a polynucleotide having at least about 60% nucleic acid sequence identity, more preferably at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% nucleic acid sequence identity and yet more preferably at least about 99% nucleic acid sequence identity with the nucleic acid sequence of SEQ ID NOs:1-12. Variants do not encompass the native nucleotide sequence.

Ordinarily, variant polynucleotides are at least about 8 nucleotides in length, often at least about 9, 10, 11, 12, 13, 14, 15, 16, 17, 18, 19, 20, 21, 22, 23, 24, 25, 26, 27, 28, 29 30, 35, 40, 45, 50, 55, 60 nucleotides in length, or even about 75-200 nucleotides in length, or more.

“Percent (%) nucleic acid sequence identity” with respect to nucleic acid sequences is defined as the percentage of nucleotides in a candidate sequence that are identical with the nucleotides in the sequence of interest, after aligning the sequences and introducing gaps, if necessary, to achieve the maximum percent sequence identity. Alignment for purposes of determining % nucleic acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

When nucleotide sequences are aligned, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) can be calculated as follows:

% nucleic acid sequence identity=W/Z*100

where

W is the number of nucleotides scored as identical matches by the sequence alignment program's or algorithm's alignment of C and D

and

Z is the total number of nucleotides in D.

When the length of nucleic acid sequence C is not equal to the length of nucleic acid sequence D, the % nucleic acid sequence identity of C to D will not equal the % nucleic acid sequence identity of D to C.

“Consisting essentially of a polynucleotide having a % sequence identity” means that the polynucleotide does not substantially differ in length, but may differ substantially in sequence. Thus, a polynucleotide “A” consisting essentially of a polynucleotide having at least 80% sequence identity to a known sequence “B” of 100 nucleotides means that polynucleotide “A” is about 100 nts long, but up to 20 nts can vary from the “B” sequence. The polynucleotide sequence in question can be longer or shorter due to modification of the termini, such as, for example, the addition of 1-15 nucleotides to produce specific types of probes, primers and other molecular tools, etc., such as the case of when substantially non-identical sequences are added to create intended secondary structures. Such non-identical nucleotides are not considered in the calculation of sequence identity when the sequence is modified by “consisting essentially of”

The specificity of single stranded DNA to hybridize complementary fragments is determined by the stringency of the reaction conditions. Hybridization stringency increases as the propensity to form DNA duplexes decreases. In nucleic acid hybridization reactions, the stringency can be chosen to favor specific hybridizations (high stringency). Less-specific hybridizations (low stringency) can be used to identify related, but not exact, DNA molecules (homologous, but not identical) or segments.

DNA duplexes are stabilized by: (1) the number of complementary base pairs, (2) the type of base pairs, (3) salt concentration (ionic strength) of the reaction mixture, (4) the temperature of the reaction, and (5) the presence of certain organic solvents, such as formamide, which decrease DNA duplex stability. A common approach is to vary the temperature: higher relative temperatures result in more stringent reaction conditions. Ausubel et al. provide an excellent explanation of stringency of hybridization reactions (Ausubel et al., 1987).

Hybridization under “stringent conditions” means hybridization protocols in which nucleotide sequences at least 60% homologous to each other remain hybridized.

Polynucleotides can include other appended groups such as peptides (e.g., for targeting host cell receptors in vivo), or agents facilitating transport across the cell membrane. In addition, oligonucleotides can be modified with hybridization-triggered cleavage agents (van der Krol et al., 1988) or intercalating agents (Zon, 1988). The oligonucleotide can be conjugated to another molecule, e.g., a peptide, a hybridization triggered cross-linking agent, a transport agent, a hybridization-triggered cleavage agent, and the like.

Useful polynucleotide analogues include polymers having modified backbones or non-natural inter-nucleoside linkages. Modified backbones include those retaining a phosphorus atom in the backbone, such as phosphorothioates, chiral phosphorothioates, phosphorodithioates, phosphotriesters, aminoalkylphosphotriesters, methyl and other alkyl phosphonates, as well as those no longer having a phosphorus atom, such as backbones formed by short chain alkyl or cycloalkyl inter-nucleoside linkages, mixed heteroatom and alkyl or cycloalkyl inter-nucleoside linkages, or one or more short chain heteroatomic or heterocyclic inter-nucleoside linkages. Modified nucleic acid polymers (analogues) can contain one or more modified sugar moieties.

Analogs that are RNA or DNA mimetics, in which both the sugar and the inter-nucleoside linkage of the nucleotide units are replaced with novel groups, are also useful. In these mimetics, the base units are maintained for hybridization with the target sequence. An example of such a mimetic, which has been shown to have excellent hybridization properties, is a peptide nucleic acid (PNA) (Buchardt et al., 1992; Nielsen et al., 1991). Another example is a locked nucleic acids (LNA) where the 2′ and 4′ glycosidic carbons are linked by a 2′-O-methylene bridge.

The realm of nucleotides includes derivatives wherein the nucleic acid molecule has been covalently modified by substitution, chemical, enzymatic, or other appropriate means with a moiety other than a naturally occurring nucleotide.

The polynucleotides of the present invention thus comprise primers that specifically hybridize to target sequences, for example the nucleic acid molecules having any one of the nucleic acid sequences of SEQ ID NOs:1-12, including analogues and/or derivatives of the nucleic acid sequences, and homologs thereof. The polynucleotides of the invention can be used as primers to amplify or detect HIV variant polynucleotides.

The polynucleotides of SEQ ID NOs:1-12 can be prepared by conventional techniques, such as solid-phase synthesis using commercially available equipment, such as that available from Applied Biosystems USA Inc. (Foster City, Calif.; USA), DuPont, (Wilmington, Del.; USA), or Milligen (Bedford, Mass.; USA). Modified polynucleotides, such as phosphorothioates and alkylated derivatives, can also be readily prepared by similar methods known in the art (Fino, 1995; Mattingly, 1995; Ruth, 1990).

### PRACTICING THE INVENTION

The invention includes methods of detecting HIV variant nucleic acids wherein a test sample is collected; reverse transcription reagents and primers, such as those of SEQ ID NO:1-12 are added, and then after reverse transcription, amplification using reagents and HIV-specific primers, such as those of SEQ ID NOs 1-12, is accomplished; the amplified nucleic acid (amplicon), if any, is analyzed using mass spectrometry; and the resulting data used to interrogate a database.

Amplification of HIV Nucleic Acids

The polynucleotides of SEQ ID NOs:1-12 can be used as primers to amplify HIV polynucleotides in a sample. The polynucleotides are used as primers, wherein the primer pairs for amplification are Set A, SEQ ID NOs:1 and 2; Set B, SEQ ID NOs:3 and 4; Set C, SEQ ID NOs:5 and 6; Set D, SEQ ID NOs:7 and 8; Set E, SEQ ID NOs:9 and 10; and Set F, SEQ ID NOs:11 and 12.

Before direct amplification with primer pair sets A-F, the template HIV polynucleotides are reverse transcribed. In one embodiment, a primer pair set selected from the group A-F is used in the reverse transcription; in another embodiment, a polyT primer is used.

The amplification method generally comprises (a) a reaction mixture comprising nucleic acid amplification reagents, at least one primer set selected from the group consisting of sets A-F, and a test sample suspected of containing at least one target sequence; and (b) subjecting the mixture to amplification conditions to generate at least one copy of a nucleic acid sequence complementary to the target sequence if the target sequence is present.

Step (b) of the above method can be repeated any suitable number of times prior to, for example, a detection step; e.g., by thermal cycling the reaction mixture between 10 and 100 times (or more), typically between about 20 and about 60 times, more typically between about 25 and about 45 times.

Nucleic acid amplification reagents include enzymes having polymerase activity, enzyme co-factors, such as magnesium or manganese; salts; nicotinamide adenine dinucleotide (NAD); and deoxynucleotide triphosphates (dNTPs), (dATP, dGTP, dCTP and dTTP).

Amplification conditions are those that promote annealing and extension of one or more nucleic acid sequences. Such annealing is dependent in a rather predictable manner on several parameters, including temperature, ionic strength, sequence length, complementarity, and G:C content of the sequences. For example, lowering the temperature in the environment of complementary nucleic acid sequences promotes annealing. Typically, diagnostic applications use hybridization temperatures that are about 2° C. to 18° C. (e.g., approximately 10° C.) below the melting temperature, Tₘ. Ionic strength also impacts Tₘ. Typical salt concentrations depend on the nature and valency of the cation but are readily understood by those skilled in the art. Similarly, high G:C content and increased sequence length stabilize duplex formation.

Finally, the hybridization temperature is selected close to or at the Tₘ of the primers. Thus, obtaining suitable hybridization conditions for a particular primer set is within the ordinary skill of the PCR arts.

Amplification procedures are well-known in the art and include the polymerase chain reaction (PCR), transcription-mediated amplification (TMA), rolling circle amplification, nucleic acid sequence based amplification (NASBA), ligase chain reaction and strand displacement amplification (SDA). One skilled in the art understands that for use in certain amplification techniques, the primers may need to be modified; for example, SDA primers usually comprise additional nucleotides near the 5′ ends that constitute a recognition site for a restriction endonuclease. For NASBA, the primers can include additional nucleotides near the 5′ end that constitute an RNA polymerase promoter. Polynucleotides thus modified are considered to be within the scope of the present invention.

The present invention includes the use of the polynucleotides of SEQ ID NOs:1-12 in methods to specifically amplify target nucleic acid sequences in a test sample.

Chemical Modification of Primers

Primers can be chemically modified, for example, to improve the efficiency of hybridization. For example, because variation (due to codon wobble in the 3^{rd} position) in conserved regions among species often occurs in the third position of a DNA triplet, the primers of SEQ ID NOs:1-12 can be modified such that the nucleotide corresponding to this position is a “universal base” that can bind to more than one nucleotide. For example, inosine (I) binds to U, C or A; guanine (G) binds to U or C, and uridine (U) binds to A or G. Other examples of universal bases include nitroindoles such as 5-nitroindole or 3-nitropyrrole (Loakes et al., 1995), the degenerate nucleotides dP or dK (Hill et al.), an acyclic nucleoside analog containing 5-nitroindazole (Van Aerschot et al., 1995) or the purine analog 1-(2-deoxy-β-D-ribofuranosyl)-imidazole-4-carboxamide (Sala et al., 1996).

In another embodiment, to compensate for the somewhat weaker binding by the “wobble” base, the oligonucleotide primers can be designed such that the first and second positions of each triplet are occupied by nucleotide analogs which bind with greater affinity than the unmodified nucleotide. Examples of these analogs include 2,6-diaminopurine, which binds to thymine; propyne T, which binds to adenine; and propyne C and phenoxazines, including G-clamp, which bind to G. Propynylated pyrimidines are described in U.S. Pat. Nos. 5,645,985, 5,830,653 and 5,484,908. Phenoxazines are described in U.S. Pat. Nos. 5,502,177, 5,763,588, and 6,005,096. G-clamps are described in U.S. Pat. Nos. 6,007,992 and 6,028,183.

Controls

Various controls can be instituted in the methods of the invention to assure, for example, that amplification conditions are optimal. An internal standard can be included in the reaction. Such internal standards generally comprise a control target nucleic acid sequence. The internal standard can optionally further include an additional pair of primers. The primary sequence of these control primers can be unrelated to the polynucleotides of the present invention and specific for the control target nucleic acid sequence.

In the context of the present invention, a control target nucleic acid sequence is a nucleic acid sequence that:

(a) can be amplified either by a primer or primer pair being used in a particular reaction or by distinct control primers; and

(b) is detected by mass spectrometric techniques.

Mass Spectrometric Characterization of Amplicons

Mass spectrometry (MS)-based detection and characterizing PCR products has several distinct advantages. MS is intrinsically a parallel detection scheme without the need for radioactive or fluorescent labels, since every amplification product is identified by its molecular mass. Less than femtomole quantities of material are required. An accurate assessment of the molecular mass of a sample can be quickly obtained. Intact molecular ions can be generated from amplification products using one of a variety of ionization techniques to convert the sample to gas phase. These ionization methods include electrospray ionization (ES), matrix-assisted laser desorption ionization (MALDI) and fast atom bombardment (FAB). For example, MALDI of nucleic acids, along with examples of matrices for use in MALDI of nucleic acids, are described in WO 98/54751.

Upon ionization, several peaks are observed from one sample due to the formation of ions with different charges. Averaging the multiple readings of molecular mass obtained from a single mass spectrum affords an estimate of molecular mass of the amplicon. Electrospray ionization mass spectrometry (ESI-MS) is particularly useful for very high molecular weight polymers such as proteins and nucleic acids having molecular weights greater than 10 kDa, since it yields a distribution of multiply-charged molecules of the sample without causing a significant amount of fragmentation.

Suitable mass detectors for the present invention include Fourier transform ion cyclotron resonance mass spectrometry (FT-ICR-MS), ion trap, quadrupole, magnetic sector, time of flight (TOF), Q-TOF, and triple quadrupole.

In general, useful mass spectrometric techniques that can be used in the present invention include tandem mass spectrometry, infrared multiphoton dissociation and pyrolytic gas chromatography mass spectrometry (PGC-MS).

The accurate measurement of molecular mass for large DNAs is limited by the adduction of cations from the PCR reaction to each strand, resolution of the isotopic peaks from natural abundance ¹³C and ¹⁵N isotopes, and assignment of the charge state for any ion. The cations are removed by in-line dialysis using a flow-through chip that brings the solution containing the PCR products into contact with a solution containing ammonium acetate in the presence of an electric field gradient orthogonal to the flow. The latter two problems can be addressed by operating with a resolving power of >100,000 and by incorporating isotopically-depleted nucleotide triphosphates into the DNA. The resolving power of the instrument is also a consideration. At a resolving power of 10,000, the modeled signal from the [M−14H+]¹⁴⁻ charge state of an 84-mer PCR product is poorly characterized and assignment of the charge state or exact mass is impossible. At a resolving power of 33,000, the peaks from the individual isotopic components are visible. At a resolving power of 100,000, the isotopic peaks are resolved to the baseline and assignment of the charge state for the ion is straightforward. The [¹³C, ¹⁵M-depleted triphosphates are obtained, for example, by growing microorganisms on depleted media and harvesting the nucleotides (Batey et al., 1992).

Tandem mass spectrometry techniques can provide more definitive information pertaining to molecular identity or sequence. Tandem MS involves the coupled use of two or more stages of mass analysis where both the separation and detection steps are based on mass spectrometry. The first stage is used to select an ion or component of a sample from which further structural information is to be obtained. The selected ion is then fragmented using, e.g., blackbody irradiation, infrared multiphoton dissociation, or collisional activation. For example, ions generated by electrospray ionization (ESI) can be fragmented using IR multiphoton dissociation. This activation leads to dissociation of glycosidic bonds and the phosphate backbone, producing two series of fragment ions, called the w-series (having an intact 3′ terminus and a 5′ phosphate following internal cleavage) and the a-Base series (having an intact 5′ terminus and a 3′ furan).

The second stage of mass analysis is then used to detect and measure the mass of these resulting fragments of product ions. Such ion selection followed by fragmentation routines can be performed multiple times so as to essentially completely dissect the molecular sequence of a sample.

PCR amplicons when analyzed by ESI-TOF mass spectrometry give a pair of masses, one for each strand of the double-stranded DNA amplicon. In some cases, the molecular mass of one strand alone provides enough information to unambiguously identify a given HIV subtype. In other cases, however, determining information from both strands is preferred.

The molecular mass of a single strand can also be consistent with more than on BCS. This can also be true for the complementary strand. These ambiguities are resolved when the added constraint of complementarity is applied. Thus a strand with a BCS of A₂₈T₂₄G₂₉C₂₅ is paired with its complement A₂₄T₂₈G₂₅C₂₉. Typically when sets of possible BCS solutions for the two strands of an amplicon are compared, usually only one pair of strands are complements of each other. That pair represents a unique solution for an amplicon's BCS; the other potential solutions are discarded because they are non-complementary.

For example, an amplicon is analyzed by ESI-TOF mass spectrometry that gives two masses: a first mass of 32,889.45 Da for one strand, and a second mass of 33,071.46 Da for the second. Assuming an average mass for the DNA bases are as follows:
A=313.0576 amu
G=328.0526 amu
C=289.0464 amu; and
T=304.0461 amu.
   Each strand has 5 possible solutions, each solution resulting in the measured mass. The calculated possible solutions for the first and second strands are:

| First Strand (32,889.45 Da) | Second strand (33,071.46 Da) |
|---|---|
| A₂₄G₂₇C₂₇T₂₄ | A₂₅G₂₆C₃₀T₂₅ |
| A₂₈G₃₁C₂₇T₂₄ | A₂₄G₂₅C₂₉T₂₈ |
| A₂₆G₃₀C₂₅T₂₅ | A₂₅G₂₅C₃₀T₂₆ |
| A₂₈G₂₉C₂₅T₂₄ | A₂₄G₂₇C₃₁T₂₈ |
| A₂₅G₃₀C₂₆T₂₅ | A₂₄G₂₇C₂₇T₂₄ |

Inspecting these possible solutions, there is only one solution where the first strand is the complement of the second strand when the constraint of complementarity is applied; that is, for every A in the first strand, there is a T in the second; for every G in the first strand, there is a C in the second, and so on. Thus, the only solution is:
First strand: A₂₈G₂₉C₂₅T₂₄
Second strand: T₂₈C₂₉G₂₅A₂₄

Mass-modifying “tags” can also be used. A nucleotide analog or “tag” is incorporated during amplification (e.g., a 5-(trifluoromethyl)deoxythymidine triphosphate) that has a different molecular weight than the unmodified base so as to improve distinction of masses. Such tags are described in, for example, WO97/33000. This further limits the number of possible base compositions consistent with any mass. For example, 5-(trifluoromethyl)deoxythymidine triphosphate can be used in place of dTTP in a separate nucleic acid amplification reaction. Measurement of the mass shift between a conventional amplification product and the tagged product is used to quantitate the number of thymidine nucleotides in each of the single strands. Because the strands are complementary, the number of adenosine nucleotides in each strand is also determined.

In another amplification reaction, the number of G and C residues in each strand is determined using, for example, the cytidine analog 5-methylcytosine (5-meC) or propyne C. The combination of the A/T reaction and G/C reaction, followed by molecular weight determination, provides a unique base composition. This method is summarized in Table 4.

TABLE 4

| | Double strand | Single strand | Total mass | Base info | Base info | Total base | Total base |
|---|---|---|---|---|---|---|---|
| Mass tag | sequence | sequence | (this strand) | (this strand) | (other strand) | comp. (top) | comp. (bottom) |
| T*mass | T*ACGT*ACGT* | T*ACGT*ACGT* | 3x | 3T | 3A | 3T | 3A |
| ( T* − T) = x | AT*GCAT*GCA | AT*GCAT*GCA | 2x | 2T | 2A | 2A | 2T |
| C*mass | TAC*GTAC*GT | TAC*GTAC*GT | 2y | 2T | 2G | 2C | 2G |
| (C* − C) = y | ATGC*ATGC*A | ATGC*ATGC*A | 2y | 2C | 2G | 2G | 2C |

In Table 4, the mass tag phosphorothioate A (A*) was used to distinguish a Bacillus anthracis cluster. The B. anthracis (A₁₄G₉C₁₄T₉) had an average MW of 14072.26, and the B. anthracis (A₁A*₁₃G₉C₁₄T₉) had an average molecular weight of 14281.11 and the phosphorothioate A had an average molecular weight of +16.06 as determined by ESI-TOF MS.

In another example, assume the measured molecular masses of each strand are 30,000.115 Da and 31,000.115 Da respectively, and the measured number of dT and dA residues are (30,28) and (28,30). If the molecular mass is accurate to 100 ppm, there are 7 possible combinations of dG+dC possible for each strand. However, if the measured molecular mass is accurate to 10 ppm, there are only 2 combinations of dG+dC, and at 1 ppm accuracy there is only one possible base composition for each strand.

Base Composition Signatures as Indices of Identifying Amplicons and Database Interrogation

Conversion of molecular mass data to a base composition signature is useful for certain analyses. A “base composition signature” (BCS) is the exact base composition determined from the molecular mass of an amplicon. The BCS can provide an index of a specific gene in a specific organism.

Base compositions, like sequences, vary slightly from isolate to isolate within species. It is possible to manage this diversity by building “base composition probability clouds” around the composition constraints for each HIV subtype. This permits identification of the subtype of HIV in a fashion similar to sequence analysis. A “pseudo four-dimensional plot” can be used to visualize the concept of base composition probability clouds. See, for example, U.S. Pat. Nos. 7,217,510, 7,108,974, 7,255,992, 7,226,739 and US 2004/0219517.

The BCS's collected from mass spectrometric analysis can be used to query a database that contains, for example, the information from the sequences amplified by primer pair sets A-F of various HIV subtypes, including G+C content, molecular mass, and of course, the BCS's, etc. From this interrogation, the subtype of HIV can be identified from the amplified target sequence. See, for example, U.S. Pat. Nos. 7,217,510, 7,108,974, 7,255,992, 7,226,739 and US 2004/0219517.

Databases

Variable regions flanked by conserved sequences, such as those flanked by primer sets A-F, can be used to build a database of BCS's. The strategy involves creating a structure-based alignment of sequences of the V3 loop variants of HIV gp120 envelope protein. Databases can also be assembled by surveying a number of HIV isolates from the field, using the primer sets A-F. In another embodiment, databases combine data from known sequences and data collected from the field.

Databases useful for the invention contain known HIV variant molecular masses and BCS's of the targeted sequences (as defined by the primer sets A-F) and, optionally, BCS's and masses from homologous regions from benign background organisms. The latter is used to estimate and subtract the signature produced by the background organisms. Optionally, a maximum likelihood detection of known background organisms is implemented using matched filters and a running-sum estimate of the noise covariance. Background signal strengths are estimated and used along with the matched filters to form signatures which are then subtracted. The maximum likelihood process is applied to this “cleaned up” data in a similar manner employing matched filters for the organisms and a running-sum estimate of the noise-covariance for the cleaned up data.

An example of data that could be compiled into a simple database for HIV-1 using primer pair sets A-C is shown in Table 5, which shows the BCS's, G+C content and molecular mass, as derived from the amplified sequences from the primer sets A-C (resulting in the sequences of SEQ ID NOs:14-16; note that the amplified sequences include the primer sequences themselves).

TABLE 5
Data from Amplified Target Sequences

| SEQ | BCS (%¹) | | | | G + C | Average |
|---|---|---|---|---|---|---|
| ID NO: | A | T | G | C | content (%) | Molecular Mass |
| 14 | 46.3 | 14.8 | 16.7 | 2.4 | 37.0 | 32913.06 |
| 15 | 47.5 | 12.9 | 16.8 | 20.8 | 37.6 | 30755.72 |
| 16 | 45.9 | 16.2 | 16.2 | 19.8 | 36.0 | 34995.62 |

¹Rounded to the nearest 0.1.

Kits

The polynucleotides of SEQ ID NOs:1-12 can be included as part of kits that allow for the detection of HIV subtype nucleic acids. Such kits comprise one or more of the polynucleotides of the invention. In one embodiment, the polynucleotides are provided in the kits in combinations for use as primers to specifically amplify HIV subtype nucleic acids in a test sample.

Kits for the detection of HIV subtype nucleic acids can also include a control target nucleic acid. Kits can also include control primers, which specifically amplify a sequence of the control target nucleic acid sequence.

Kits can also include amplification reagents, reaction components and/or reaction vessels. One or more of the polynucleotides can be modified as previously discussed. One or more of the components of the kit may be lyophilized, and the kit can further include reagents suitable for reconstituting the lyophilized products. The kit can additionally contain instructions for use.

In an additional embodiment, the kits further contain computer-readable media that contains a database that allows for the identification of BCS's. Optionally, the computer-readable media can contain software that allows for data collection and/or database interrogation. Kits can also be supplied with instructional materials. Instructions may be printed on paper or other substrate, and/or may be supplied as an electronic-readable medium, such as a floppy disc, CD-ROM, DVD-ROM, Zip disc, videotape, audiotape, etc.

When a kit is supplied, the different components of the composition can be packaged in separate containers and admixed immediately before use. Such packaging of the components separately can permit long-term storage of the active components. For example, one or more of the particles having polynucleotides attached thereto, the substrate, and the nucleic acid enzyme are supplied in separate containers.

The reagents included in the kits can be supplied in containers of any sort such that the different components are preserved and are not adsorbed or altered by the materials of the container. For example, sealed glass ampoules can contain one of more of the reagents or buffers that have been packaged under a neutral, non-reacting gas, such as nitrogen. Ampoules can consist of any suitable material, such as glass, organic polymers, such as polycarbonate, polystyrene, etc.; ceramic, metal or any other material typically used to hold similar reagents. Other examples of suitable containers include simple bottles that can be fabricated from similar substances as ampoules, and envelopes, that can have foil-lined interiors, such as aluminum or an alloy. Other containers include test tubes, vials, flasks, bottles, syringes, etc.

### EXAMPLES

The following examples are for illustrative purposes only and should not be interpreted as limitations of the claimed invention. There are a variety of alternative techniques and procedures available to those of skill in the art which would similarly permit one to successfully perform the intended invention.

### Example 1

### Primer Design

In this example, the A-F amplification primer sets suitable for use polymerase chain reactions and other polynucleotide amplification protocols were designed to produce amplification products that were suitable for mass spectrometric analysis to allow identification of V3 loop variants of HIV gp120 envelope protein involved in T3-cell/macrophage tropism.

The primers were designed using OLIGO 6 software (Molecular Biology Insights, Inc.; Cascade, Colo.), using the following design parameters:

200 nM primer concentration

50 mM monovalent cation

0.7 mM free divalent cation

### Example 2

### Demonstration of Primer Efficacy to Amplify Target Sequence: Primer Sets A, B and C (Prophetic)

Primer sets, A (SEQ ID NOs:1 and 2) and B (SEQ ID NOs:3 and 4) and C (SEQ ID NOs:5 and 6), as shown in Table 1 and reproduced in Table 6, are tested for their ability to amplify the target sequence and for the amplified sequence to be detected. The primers themselves can further incorporate a nucleotide analog, such as inosine, uridine, 2,6-diaminopurine, propyne C or propyne T. The amplification reaction is preceded by a reverse transcription reaction to create a cDNA template from the HIV RNA genome.

TABLE 6
Primer sequences

| | | SEQ | | Ampli- |
|---|---|---|---|---|
| | | ID | | con |
| Primer | Sequence | NO | Tm | length* |
| A (Forward) | aagacccaac aacaatacaa | 1 | 65° | |
| | ga | | | |
| A (Reverse) | gttacaatgt gcttgtctca | 2 | 65° | 106 |
| | tattt | | | |
| B (Forward) | aagacccaac aacaatacaa | 3 | 66° | |
| | gaa | | | |
| B (Reverse) | tgtgcttgtc tcatatttcc | 4 | 67° | 99 |
| | tattt | | | |
| C (Forward) | taattgtaca agacccaaca | 5 | 66° | |
| | aca | | | |
| C (Reverse) | atgtgcttgt ctcatatttc | 6 | 66° | 109 |
| | ctatt | | | |
| D (Forward) | gacccaacaa caatacaaga | 7 | 64° | |
| | a | | | |
| D (Reverse) | tgtgcttgtc ttatatctcc | 8 | 63° | 94 |
| | tatta | | | |
| E (Forward) | ttgtacaaga cccaacaaca | 9 | 64° | |
| E (Reverse) | aatgtgcttg tcttatatct | 10 | 63° | 104 |
| | cctat | | | |
| F (Forward) | attaattgta caagacccaa | 11 | 62° | |
| | ca | | | |
| F (Reverse) | atgtgcttgt cttatatctc | 12 | 63° | 108 |
| | ctatt | | | |

*Amplicon length includes primer sequences.

The conditions for the PCR are:

| | | | |
|---|---|---|---|
| 94° | C. | 1 × 10 sec. | 35 cycles |
| 55-60° | C. | 1 × 20 sec. | |
| 72° | C. | 1 × 20 sec. | |
| 4° | C. | hold | 1 cycle |

Alternatively, a “hot start” polymerase can be used in order to avoid false priming during the initial rounds of PCR. Adjustments to the PCR cycling parameters would include a heat activation step prior to the standard PCR cycling.

| | | | |
|---|---|---|---|
| 94° | C. | 5-10 min. | 1 cycle |
| 94° | C. | 1 × 10 sec. | 35 cycles |
| 55-60° | C. | 1 × 20 sec. | |
| 72° | C. | 1 × 20 sec. | |
| 4° | C. | hold | 1 cycle |

The predicted amplification products are subjected to mass spectrometric analysis, their BCS determined and coupled with database interrogation, wherein the database contains the BCS information from the regions targeted by the primer sets A (SEQ ID NOs:1 and 2), B (SEQ ID NOs:3 and 4), and C (SEQ ID NOs:5 and 6), including the mass and/or BCS of each target sequence. This protocol can also be carried out with primer sets D, E, and F.

### Example 3

### Demonstration of Primer Efficacy to Amplify Target Sequence: Primer Sets D, E, and F (Prophetic)

Primer sets D (SEQ ID NOs:7 and 8) and E (SEQ ID NOs:9 and 10) and F (SEQ ID NOs:11 and 12), as shown in Table 1 and reproduced in Table 6, are tested for their ability to amplify the target sequence and for the amplified sequence to be detected, wherein the amplification reaction is divided into two stages: a first stage where amplification is performed under low stringency PCR conditions, and then a second stage under high stringency PCR conditions. The low stringency allows for base pair mis-matches between the primer sequence and the HIV cDNA sequence to allow for primer hybridization to different HIV subtypes. The primers themselves can further incorporate a nucleotide analog, such as inosine, uridine, 2,6-diaminopurine, propyne C or propyne T. The amplification reaction is preceded by a reverse transcription reaction to create a cDNA template from the HIV RNA genome.

The cycle for the low stringency PCR is:

| | | | |
|---|---|---|---|
| 94° | C. | 1 × 10 sec. | 5 cycles |
| 40-50° | C. | 1 × 20 sec. | |
| 72° | C. | 1 × 20 sec. | |

The high stringency PCR cycle is:

| | | | |
|---|---|---|---|
| 94° | C. | 1 × 10 sec. | 30 cycles |
| 55-60° | C. | 1 × 20 sec. | |
| 72° | C. | 1 × 20 sec. | |
| 4° | C. | hold | 1 cycle |

The predicted amplification products are subjected to mass spectrometric analysis, their BCS determined and coupled with database interrogation, wherein the database contains the BCS information from the regions targeted by the primer sets A (SEQ ID NOs:7 and 8), B (SEQ ID NOs:9 and 10), and C (SEQ ID NOs:11 and 12), including the mass and/or BCS of each target sequence. This protocol can also be carried out with primer sets A, B, and C.

### Example 4

### Mass Spectrometry (Prophetic)

Fourier transform ion cyclotron resonance (FTICR) mass spectrometry Instrumentation: The FT-ICR instrument is based on a 7 tesla actively shielded superconducting magnet and modified Bruker Daltonics Apex II 70e ion optics (Bruker Daltonics; Billerica; MA) and vacuum chamber. The spectrometer is interfaced to a LEAP PAL autosampler (LEAP Technologies; Carrboro, N.C.) and a custom fluidics control system for high throughput screening applications. Samples are analyzed directly from 96-well or 384-well microtiter plates at a rate of about 1 sample/minute. The Bruker data-acquisition platform is supplemented with a lab-built ancillary data station that controls the autosampler and contains an arbitrary waveform generator capable of generating complex rf-excite waveforms (frequency sweeps, filtered noise, stored waveform inverse Fourier transform (SWIFT), etc.) for tandem MS experiments. Typical performance characteristics include mass resolving power in excess of 100,000 (FWHM), low ppm mass measurement errors, and an operable m/z range between 50 and 5000 m/z.

Modified ESI Source: In sample-limited analyses, analyte solutions are delivered at 150 mL/minute to a 30 mm i.d. fused-silica ESI emitter mounted on a 3-D micromanipulator. The ESI ion optics consists of a heated metal capillary, an rf-only hexapole, a skimmer cone, and an auxiliary gate electrode. The 6.2 cm rf-only hexapole is comprised of 1 mm diameter rods and is operated at a voltage of 380 Vpp at a frequency of 5 MHz. An electro-mechanical shutter can be used to prevent the electrospray plume from entering the inlet capillary unless triggered to the “open” position via a TTL pulse from the data station. When in the “closed” position, a stable electrospray plume is maintained between the ESI emitter and the face of the shutter. The back face of the shutter arm contains an elastomeric seal that can be positioned to form a vacuum seal with the inlet capillary. When the seal is removed, a 1 mm gap between the shutter blade and the capillary inlet allows constant pressure in the external ion reservoir regardless of whether the shutter is in the open or closed position. When the shutter is triggered, a “time slice” of ions is allowed to enter the inlet capillary and is subsequently accumulated in the external ion reservoir. The rapid response time of the ion shutter (<25 ms) provides reproducible, user defined intervals during which ions can be injected into and accumulated in the external ion reservoir.

Apparatus for Infrared Multiphoton Dissociation: A 25-watt CW CO₂ laser operating at 10.6 μm is interfaced to the spectrometer to enable infrared multiphoton dissociation (IRMPD) for tandem MS applications. An aluminum optical bench is positioned approximately 1.5 m from the actively shielded superconducting magnet such that the laser beam is aligned with the central axis of the magnet. Using standard infrared-compatible mirrors and kinematic mirror mounts, the unfocused 3 mm laser beam is aligned to traverse directly through the 3.5 mm holes in the trapping electrodes of the FT-ICR trapped ion cell and longitudinally traverse the hexapole region of the external ion guide finally impinging on the skimmer cone. This scheme allows infrared multiphoton dissociation (IRMPD) to be conducted in an m/z selective manner in the trapped ion cell (e.g. following a SWIFT isolation of the species of interest), or in a broadband mode in the high pressure region of the external ion reservoir where collisions with neutral molecules stabilize IRMPD-generated metastable fragment ions resulting in increased fragment ion yield and sequence coverage.

### Example 5

### Assaying for the Presence of HIV from a Test Sample (Prophetic)

A sample from an organism or subject suspected of carrying HIV is processed using well-known methods and following reverse transcription, is assayed using a primer set selected from the group consisting of A-F using PCR using standard methods, such as those shown in Examples 2 or 3. The amplified products are assayed by mass spectrometry using the set-up described in Example 4.

If necessary, nucleic acid is isolated from the samples, for example, by cell lysis, centrifugation and ethanol precipitation or any other technique well known in the art.

Mass measurement accuracy can be assayed using an internal mass standard in the ESI-MS study of PCR products. A mass standard, such as a 20-mer phosphorothioate oligonucleotide added to a solution containing a primer set A-E and/or F PCR product(s) from HIV.

The predicted amplification products are subjected to mass spectrometric analysis coupled with database interrogation, wherein the database contains the information from the regions targeted by the primer sets A-F, including the mass and/or BCS of each target sequence.

### REFERENCES

Aaserud, D. J., N. L. Kelleher, D. P. Little, et al. 1996. Accurate base composition of double-strand DNA by mass spectrometry. J. Am. Soc. Mass Spec. 7:1266-1269.
Ausubel, F. M., R. Brent, R. E. Kingston, et al. 1987. Current protocols in molecular biology. John Wiley & Sons, New York.

Batey, R.T., M. Inada, E. Kujawinski, et al. 1992. Preparation of isotopically labeled ribonucleotides for multidimensional NMR spectroscopy of RNA. Nucleic Acids Res. 20:4515-23.
Buchardt, O., P. Nielsen, and R. Berg. 1992. PEPTIDE NUCLEIC ACIDS.
Clapham, P. R., and A. McKnight. 2001. HIV-1 receptors and cell tropism. Br Med. Bull. 58:43-59.
Fino, J. U.S. Pat. No. 5,464,746. 1995. HAPTENS, TRACERS, IMMUNOGENS AND ANTIBODIES FOR CARBAZOLE AND DIBENZOFURAN DERIVATIVES.
Gao, F., S. G. Morrison, D. L. Robertson, et al. 1996. Molecular cloning and analysis of functional envelope genes from human immunodeficiency virus type 1 sequence subtypes A through G. The WHO and NIAID Networks for HIV Isolation and Characterization. J. Virol. 70:1651-67.
Hurst, G. B., M. J. Doktycz, A. A. Vass, et al. 1996. Detection of bacterial DNA polymerase chain reaction products by matrix-assisted laser desorption/ionization mass spectrometry. Rapid Commun Mass Spectrom. 10:377-82.
Loakes, D., F. Hill, S. Linde, et al. 1995. Nitroindoles as universal bases. Nucleosides Nucleotides. 14:1001-1003.
Low, A. J., L. C. Swenson, and P. R. Harrigan. 2008. HIV coreceptor phenotyping in the clinical setting. AIDS Rev. 10:143-51.
Mattingly, P. U.S. Pat. No. 5,424,414. 1995. HAPTENS, TRACERS, IMMUNOGENS AND ANTIBODIES FOR 3-PHENYL-A-ADAMANTANEACETIC ACIDS.
Muddiman, D., and R. Smith. 1998. Sequencing and Characterization of Larger Oligonucleotides by Electrospray Ionization Fourier Transform Ion Cyclotron Resonance Mass Spectrometry. Rev. Anal. Chem. 17:1-68.
Muddiman, D. C., G. A. Anderson, S. A. Hofstadler, et al. 1997. Length and base composition of PCR-amplified nucleic acids using mass measurements from electrospray ionization mass spectrometry. Anal Chem. 69:1543-9.
Muddiman, D. C., A. P. Null, and J. C. Hannis. 1999. Precise mass measurement of a double-stranded 500 base-pair (309 kDa) polymerase chain reaction product by negative ion electrospray ionization Fourier transform ion cyclotron resonance mass spectrometry. Rapid Commun Mass Spectrom. 13:1201-1204.
Nielsen, P. E., M. Egholm, R. H. Berg, et al. 1991. Sequence-selective recognition of DNA by strand displacement with a thymine-substituted polyamide. Science. 254:1497-500.
Robertson, D. L., P. M. Sharp, F. E. McCutchan, et al. 1995. Recombination in HIV-1. Nature. 374:124-6.
Ruth, J. U.S. Pat. No. 4,948,882. 1990. Ruth, J. 1990. SINGLE-STRANDED LABELED OLIGONUCLEOTIDES, REACTIVE MONOMERS AND METHODS OF SYNTHESIS.
Sala, M., V. Pezo, S. Pochet, et al. 1996. Ambiguous base pairing of the purine analogue 1-(2-deoxy-beta-D-ribofuranosyl)-imidazole-4-carboxamide during PCR. Nucleic Acids Res. 24:3302-6.
Van Aerschot, A., C. Hendrix, G. Schepers, et al. 1995. In search of acyclic analogs as universal nucleosides in degenerate probes. Nucleosides Nucleotides. 14:1053-1056.
Van Baelen, K., I. Vandenbroucke, E. Rondelez, et al. 2007. HIV-1 coreceptor usage determination in clinical isolates using clonal and population-based genotypic and phenotypic assays. J Virol Methods. 146:61-73.
van der Krol, A. R., J. N. Mol, and A. R. Stuitje. 1988. Modulation of eukaryotic gene expression by complementary RNA or DNA sequences. Biotechniques. 6:958-76.
Westby, M., M. Lewis, J. Whitcomb, et al. 2006. Emergence of CXCR4-using human immunodeficiency virus type 1 (HIV-1) variants in a minority of HIV-1-infected patients following treatment with the CCR5 antagonist maraviroc is from a pretreatment CXCR4-using virus reservoir. J Virol. 80:4909-20.
Wunschel, D.S., D.C. Muddiman, K. F. Fox, et al. 1998. Heterogeneity in Bacillus cereus PCR products detected by ESI-FTICR mass spectrometry. Anal Chem. 70:1203-7.
Zon, G. 1988. Oligonucleotide analogues as potential chemotherapeutic agents. Pharm Res. 5:539-49.

1. A method of identifying an HIV subtype in a test sample, comprising:
providing a test sample;
forming a reaction mixture comprising:
a primer pair set selected from the group consisting of set A, B, C, D, E, and F, wherein:
set A comprises a forward primer comprising a nucleic acid sequence of SEQ ID NO:1, and a reverse primer comprising a nucleic acid sequence of SEQ ID NO:2;
set B comprises a forward primer comprising a nucleic acid sequence of SEQ ID NO:3, and a reverse primer comprising a nucleic acid sequence of SEQ ID NO:4;
set C comprises a forward primer comprising a nucleic acid sequence of SEQ ID NO:5, and a reverse primer comprising a nucleic acid sequence of SEQ ID NO:6,
set D comprises a forward primer comprising a nucleic acid sequence of SEQ ID NO:7; and a reverse primer comprising a nucleic acid sequence of SEQ ID NO:8;
set E comprises a forward primer comprising a nucleic acid sequence of SEQ ID NO:9, and a reverse primer comprising a nucleic acid sequence of SEQ ID NO:IO; and
set F comprises a forward primer comprising a nucleic acid sequence of SEQ ID NO: 11, and a reverse primer comprising a nucleic acid sequence of SEQ ID NO:I2;
subjecting the mixture to amplification conditions to generate an amplification product;
determining the molecular mass and base composition of the amplification product; and comparing the molecular mass and base composition of the amplification product to calculated or measured molecular masses and base compositions of target sequences in a database to identify the HIV subtype

2. (canceled)

3. The method of claim 1, wherein the identifying the target sequence does not comprise sequencing of the amplification product.

4. The method of claim 1 wherein the mass spectrometry is Fourier transform ion cyclotron resonance mass spectrometry (FT-ICR-MS), time of flight mass spectrometry (TOF-MS), or electrospray ionization time of flight spectroscopy.

5. The method of claim 1, wherein the primer set comprises at least one nucleotide analog.

6. The method of claim 5, wherein the nucleotide analog is selected from the group consisting of inosine, uridine, 2,6-diaminopurine, propyne C, and propyne T.

7. The method of claim 1, wherein a molecular mass-modifying tag is incorporated into the amplification product.

8. A method of identifying an HIV subtype in a test sample, comprising:
providing a test sample;
forming a reaction mixture comprising:
a primer pair set selected from the group consisting of set A, B, C, D, E, and F, wherein:
set A comprises a forward primer comprising a nucleic acid sequence having at least 80% sequence identity with a nucleic acid sequence of SEQ ID NO:1, and a reverse primer comprising a nucleic acid sequence having at least 80% sequence identity with a nucleic acid sequence of SEQ ID NO:2;
set B comprises a forward primer comprising a nucleic acid sequence having at least 80% sequence identity with a nucleic acid sequence of SEQ ID NO:3, and a reverse primer comprising a nucleic acid sequence having at least 80% sequence identity with a nucleic acid sequence of SEQ ID NO:4;
set C comprises a forward primer comprising a nucleic acid sequence having at least 80% sequence identity with a nucleic acid sequence of SEQ ID NO:5, and a reverse primer comprising a nucleic acid sequence having at least 80% sequence identity with a nucleic acid sequence of SEQ ID NO:6;
set D comprises a forward primer comprising a nucleic acid sequence of SEQ ID NO:7, and a reverse primer comprising a nucleic acid sequence of SEQ ID NO:8;
set E comprises a forward primer comprising a nucleic acid sequence of SEQ ID NO:9, and a reverse primer comprising a nucleic acid sequence of SEQ ID NO:I0; and
set F comprises a forward primer comprising a nucleic acid sequence of SEQ ID NO: 11, and a reverse primer comprising a nucleic acid sequence of SEQ ID NO:12;
subjecting the mixture to amplification conditions to generate an amplification product;
determining the molecular mass and base composition of the amplification product; and
comparing the molecular mass and base composition of the amplification product to calculated or measured molecular masses and base compositions of target sequences in a database to identify the HIV subtype.

9. (canceled)

10. The method of claim 8, wherein the identifying the target sequence does not comprise sequencing of the amplification product.

11. The method of claim 8, wherein the mass spectrometry is Fourier transform ion cyclotron resonance mass spectrometry (FT-ICR-MS), time of flight mass spectrometry (TOF-MS), or electrospray ionization time of flight spectroscopy.

12. The method of claim 8, wherein the primer set comprises at least one nucleotide analog.

13. The method of claim 12, wherein the nucleotide analog is selected from the group consisting of inosine, uridine, 2,6-diaminopurine, propyne C, and propyne T.

14. The method of claim 9, wherein a molecular mass-modifying tag is incorporated into the amplification product.

15. A kit, comprising a primer pair set selected from the group consisting of set A, B, C, D, E, and F, wherein:
set A comprises a forward primer comprising a nucleic acid sequence of SEQ ID NO:1, and a reverse primer comprising a nucleic acid sequence of SEQ ID NO:2;
set B comprises a forward primer comprising a nucleic acid sequence of SEQ ID NO:3, and a reverse primer comprising a nucleic acid sequence of SEQ ID NO:4;
set C comprises a forward primer comprising a nucleic acid sequence of SEQ ID NO:5, and a reverse primer comprising a nucleic acid sequence of SEQ ID NO:6;
set D comprises a forward primer comprising a nucleic acid sequence of SEQ ID NO:7, and a reverse primer comprising a nucleic acid sequence of SEQ ID NO:8;
set E comprises a forward primer comprising a nucleic acid sequence of SEQ ID NO:9, and a reverse primer comprising a nucleic acid sequence of SEQ ID NO:I0; and
set F comprises a forward primer comprising a nucleic acid sequence of SEQ ID NO: 11, and a reverse primer comprising a nucleic acid sequence of SEQ ID NO: 12; and
amplification reagents.

16. The kit of claim 15, further comprising a means to access a database comprising calculated or measured base compositions or molecular masses of target sequences in a database to identify the HIV subtype.

17. A kit, comprising a primer pair set selected from the group consisting of set A, B, C, D, E, and F, wherein:
set A comprises a forward primer comprising a nucleic acid sequence having at least 80% sequence identity with a nucleic acid sequence of SEQ ID NO:1, and a reverse primer comprising a nucleic acid sequence having at least 80% sequence identity with a nucleic acid sequence of SEQ ID NO:2;
set B comprises a forward primer comprising a nucleic acid sequence having at least 80% sequence identity with a nucleic acid sequence of SEQ ID NO:3, and a reverse primer comprising a nucleic acid sequence having at least 80% sequence identity with a nucleic acid sequence of SEQ ID NO:4; and
set C comprises a forward primer comprising a nucleic acid sequence having at least 80% sequence identity with a nucleic acid sequence of SEQ ID NO:5, and a reverse primer comprising a nucleic acid sequence having at least 80% sequence identity with a nucleic acid sequence of SEQ ID NO:6;
set D comprises a forward primer comprising a nucleic acid sequence having at least 80% sequence identity with a nucleic acid sequence of SEQ ID NO:7, and a reverse primer comprising a nucleic acid sequence having at least 80% sequence identity with a nucleic acid sequence of SEQ ID NO:8;
set E comprises a forward primer comprising a nucleic acid sequence having at least 80% sequence identity with a nucleic acid sequence of SEQ ID NO:9, and a reverse primer comprising a nucleic acid sequence having at least 80% sequence identity with a nucleic acid sequence of SEQ ID NO: 10;
set F comprises a forward primer comprising a nucleic acid sequence having at least 80% sequence identity with a nucleic acid sequence of SEQ ID NO: 11, and a reverse primer comprising a nucleic acid sequence having at least 80% sequence identity with a nucleic acid sequence of SEQ ID NO: 12; and amplification reagents.

18. The kit of claim 17, further comprising a means to access a database comprising calculated or measured base compositions or molecular masses of target sequences in a database to identify the HIV subtype.

19. A method of identifying at least two HIV subtypes in a test sample, comprising:
providing a test sample;
forming a reaction mixture comprising:
a primer pair set selected from the group consisting of set A, B, C, D, E, and F, wherein:
set A comprises a forward primer comprising a nucleic acid sequence having at least 80% sequence identity with a nucleic acid sequence of SEQ ID NO:1,
and a reverse primer comprising a nucleic acid sequence having at least 80% sequence identity with a nucleic acid sequence of SEQ ID NO:2;
set B comprises a forward primer comprising a nucleic acid sequence having at least 80% sequence identity with a nucleic acid sequence of SEQ ID NO:3,
and a reverse primer comprising a nucleic acid sequence having at least 80% sequence identity with a nucleic acid sequence of SEQ ID NO:4; and
set C comprises a forward primer comprising a nucleic acid sequence having at least 80% sequence identity with a nucleic acid sequence of SEQ ID NO:5, and a reverse primer comprising a nucleic acid sequence having at least 80% sequence identity with a nucleic acid sequence of SEQ ID NO:6;
set D comprises a forward primer comprising a nucleic acid sequence having at least 80% sequence identity with a nucleic acid sequence of SEQ ID NO:7, and a reverse primer c comprising a nucleic acid sequence having at least 80% sequence identity with a nucleic acid sequence of SEQ ID NO:8;
set E comprises a forward primer comprising a nucleic acid sequence having at least 80% sequence identity with a nucleic acid sequence of SEQ ID NO:9, and a reverse primer comprising a nucleic acid sequence having at least 80% sequence identity with a nucleic acid sequence of SEQ ID NO: 10; and
set F comprises a forward primer comprising a nucleic acid sequence having at least 80% sequence identity with a nucleic acid sequence of SEQ ID NO: 11, and a reverse primer comprising a nucleic acid sequence having at least 80% sequence identity with a nucleic acid sequence of SEQ ID NO: 12; subjecting the mixture to amplification conditions to generate an amplification product;
determining the molecular mass and base composition of the amplification product; and
comparing the molecular mass and base composition of the amplification product to calculated or measured molecular masses and base compositions of target sequences in a database to identify the at least two HIV subtypes.

20. (canceled)

21. The method of claim 19, wherein the at least two subtypes comprise a CCR5-binding subtype and a CXCR4-binding subtype.

22. The method of claim 21, wherein the CXCR4-binding subtype comprises at least 1% of the total HIV in the test sample.

23. The method of claim 22, wherein the CXCR4-binding subtype comprises at least 5% of the total HIV in the test sample.

24. The method of claim 22, wherein the CXCR4-binding subtype comprises at least 10% of the total HIV in the test sample.


SEQUENCE LISTING (19 sequences)

SEQ ID NO: 1 - DNA, 22 bp, Artificial Sequence
  source: /note=“Description of Artificial HG Sequence Synthetic primer”
aagacccaacaacaatacaaga

SEQ ID NO: 2 - DNA, 25 bp, Artificial Sequence
  source: /note=“Description of Artificial Sequence Synthetic primer”
gttacaatgtgcttgtctcatattt

SEQ ID NO: 3 - DNA, 23 bp, Artificial Sequence
  source: /note=“Description of Artificial Sequence Synthetic primer”
aagacccaacaacaatacaagaa

SEQ ID NO: 4 - DNA, 25 bp, Artificial Sequence
  source: /note=“Description of Artificial Sequence Synthetic primer”
tgtgcttgtctcatatttcctattt

SEQ ID NO: 5 - DNA, 23 bp, Artificial Sequence
  source: /note=“Description of Artificial Sequence Synthetic primer”
taattgtacaagacccaacaaca

SEQ ID NO: 6 - DNA, 25 bp, Artificial Sequence
  source: /note=“Description of Artificial Sequence Synthetic primer”
atgtgcttgtctcatatttcctatt

SEQ ID NO: 7 - DNA, 21 bp, Artificial Sequence
  source: /note=“Description of Artificial Sequence Synthetic primer”
gacccaacaacaatacaagaa

SEQ ID NO: 8 - DNA, 25 bp, Artificial Sequence
  source: /note=“Description of Artificial Sequence Synthetic primer”
tgtgcttgtcttatatctcctatta

SEQ ID NO: 9 - DNA, 20 bp, Artificial Sequence
  source: /note=“Description of Artificial Sequence Synthetic primer”
ttgtacaagacccaacaaca

SEQ ID NO: 10 - DNA, 25 bp, Artificial Sequence
  source: /note=“Description of Artificial Sequence Synthetic primer”
aatgtgcttgtcttatatctcctat

SEQ ID NO: 11 - DNA, 22 bp, Artificial Sequence
  source: /note=“Description of Artificial Sequence Synthetic primer”
attaattgtacaagacccaaca

SEQ ID NO: 12 - DNA, 25 bp, Artificial Sequence
  source: /note=“Description of Artificial Sequence Synthetic primer”
atgtgcttgtcttatatctcctatt

SEQ ID NO: 13 - DNA, 2571 bp, Human immunodeficiency virus 1
atgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagaatccgtatccagagaggaccagggagagcatttgttacaataggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataa

SEQ ID NO: 14 - DNA, 106 bp, Artificial Sequence
  source: /note=“Description of Artificial Sequence Synthetic primer”
aagacccaacaacaatacaagaaaaagaatccgtatccagagaggaccagggagagcatttgttacaataggaaaaataggaaatatgagacaagcacattgtaac

SEQ ID NO: 15 - DNA, 99 bp, Artificial Sequence
  source: /note=“Description of Artificial Sequence Synthetic primer”
aagacccaacaacaatacaagaaaaagaatccgtatccagagaggaccagggagagcatttgttacaataggaaaaataggaaatatgagacaagcaca

SEQ ID NO: 16 - DNA, 109 bp, Artificial Sequence
  source: /note=“Description of Artificial Sequence Synthetic primer”
taattgtacaagacccaacaacaatacaagaaaaagaatccgtatccagagaggaccagggagagcatttgttacaataggaaaaataggaaatatgagacaagcacat

SEQ ID NO: 17 - DNA, 51 bp, Artificial Sequence
  source: /note=“Description of Artificial Sequence Synthetic primer”
aaagaatccgtatccagagaggaccagggagagcatttgttacaataggaa

SEQ ID NO: 18 - DNA, 62 bp, Artificial Sequence
  source: /note=“Description of Artificial Sequence Synthetic primer”
atacaagaaaaagaatccgtatccagagaggaccagggagagcatttgttacaataggaaaa

SEQ ID NO: 19 - DNA, 64 bp, Artificial Sequence
  source: /note=“Description of Artificial Sequence Synthetic primer”
acaatacaagaaaaagaatccgtatccagagaggaccagggagagcatttgttacaataggaaa